(19) United States
(12) Patent Application Publication
(10) Pub. No.: US 2010/0178211 A1
(43) Pub. Date: Jul. 15, 2010

(54) COMMUNICATION TERMINAL DEVICE

(75) Inventor: Tetsuya Ushijima, Yokohama-shi (JP)

Correspondence Address: Christensen O'Connor Johnson Kindness PLLC, 1420 Fifth Avenue, Suite 2800, Seattle, WA, 98101-2347, US

(73) Assignee: PANASONIC CORPORATION, Kadoma-shi, Osaka (JP)

(21) Appl. No.: 12/602,961

(22) PCT Filed: Jun. 11, 2007
(86) PCT No.: PCT/JP2007/061745

§ 371 Date: Dec. 3, 2009

(51) Int. Cl. B05B 11/06; A61L 9/00; A61L 9/14; A61L 9/015; G05D 7/06; H04M 1/21

(52) U.S. Cl. 422/106; 422/114; 422/116; 422/119; 422/111

(57) ABSTRACT

A communication terminal device in which a diffusion control of aroma suitable for the situation can be performed. In the communication terminal device (100), according to the diffusion parameter used for diffusing the aroma (or smoke) and predetermined by the user, the state of a mobile terminal body (100B) recognized by a control unit (130) by using the detected information from the sensors of a sensing unit (150), and environment information on the environment around the mobile terminal body (100B), diffusion control of an aroma material (121) from a diffusion apparatus body (100A) is performed by an aroma diffusion section (141) of an indicating unit (140), smoke generation control of a smoke generation material (122) is performed by a smoke generation section (142), and suppression control of the aroma diffusion and the smoke generation is performed by a diffusion suppression unit (110). Therefore, the diffusion control of the aroma and the smoke that is suitable for the situation (environment) of the mobile terminal main body (100B) can be performed.

### TECHNICAL FIELD

The present invention relates to communication terminal apparatus, including a mobile phone having fragrance spreading functions for notifying incoming calls and schedules by spreading fragrance.

### BACKGROUND ART

In recent years, in the field of communication terminal apparatus such as mobile phones, it is becoming important to have not only the basic functions related to communication areas and communication speed, but also have an added value in an emotional aspect such as “fun” “amusing” like “ringtones” and “design.”

A mobile terminal apparatus with added value for notifying incoming calls, schedules and so on by spreading fragrance, is disclosed (see Patent Document 1). By this means, the user can recognize a notification such as an incoming call even in the situation where the user does not wear a mobile terminal apparatus. Further, a fragrance-producing mobile phone and utilization method thereof that entertains the user with fragrances according to senders of incoming calls or received email messages is disclosed (see Patent Document 2). By this means, it is possible to identify information received as input to a terminal by the sense of smell.
Patent Document 1: Japanese Patent Application Laid-Open No. 2005-318345
Patent Document 2: Japanese Patent Application Laid-Open No. 2002-252686

### DISCLOSURE OF INVENTION

### Problems to be Solved by the Invention

Many requirements for spreading fragrance are proposed for communication terminal apparatuses such as conventional mobile terminal apparatus and fragrance-producing mobile phone. However, the influence of the fragrance spreading and wafting in the air every incoming call has not been considered for the neighborhood. For this reason, with conventional communication terminal apparatuses, a spread fragrance may cause trouble with others depending upon situations.

It is therefore an object of the present invention to provide a communication terminal apparatus that enables suitable control to spread fragrance depending upon situations.

### Means for Solving the Problem

The communication terminal apparatus of the present invention adopts a configuration including: a fragrance spreading section that spreads fragrance by a predetermined spreading request; a spread parameter setting section that sets up a spread parameter upon spreading the fragrance by the fragrance spreading section; an environment information acquiring section that acquires environment information about an environment in which the fragrance spreading section spreads the fragrance; and a spread canceling section that cancels spreading the fragrance based on the spread parameter and the environment information.

### Advantageous Effects of Invention

According to the present invention, it is possible to cancel spreading fragrance based on fragrance spread parameters and information about the environment in which fragrance is spread, so that it is possible to realize a communication terminal apparatus that enables suitable control to spread fragrance depending upon situations.

### BRIEF DESCRIPTION OF DRAWINGS

FIG. 1 is a block diagram showing the configuration of a communication terminal apparatus according to an embodiment of the present invention;

FIG. 2 is a flow chart showing operations of the fragrance control requesting process in communication terminal apparatus according to an embodiment of the present invention;

FIG. 3 is a flow chart showing operations of fragrance spreading process in the communication terminal apparatus according to an embodiment of the present invention;

FIG. 4 is a flow chart showing operations of preliminary spreading process in the communication terminal apparatus according to an embodiment of the present invention;

FIG. 5 is a flow chart showing operations of spreading process in the communication terminal apparatus according to an embodiment of the present invention; and

FIG. 6 is a flow chart showing operations of fragrance spreading stop (canceling) process in the communication terminal apparatus according to an embodiment of the present invention.

### BEST MODE FOR CARRYING OUT THE INVENTION

A conventional communication terminal apparatus configured to notify an incoming call or an incoming email message by spreading fragrance, only spreads fragrance every incoming call. For this reason, with such a communication terminal apparatus, in the environments where the spread of fragrance may cause trouble with others, in a restaurant, a car, and a movie theater, for example, there has been a problem of spreading fragrance due to an incoming call.

In view of the above-described problem, the present invention enables suitable control to spread fragrance depending upon situations, by for example, canceling the spread of fragrance when a communication terminal apparatus is in an environment not suitable for spreading fragrance, the communication terminal apparatus.

Now, an embodiment of the present invention will be described in detail with reference to the accompanying drawings. Components and corresponding parts in the drawings having the same configurations or functions will be assigned the same reference numerals, and their explanations will not be repeated.

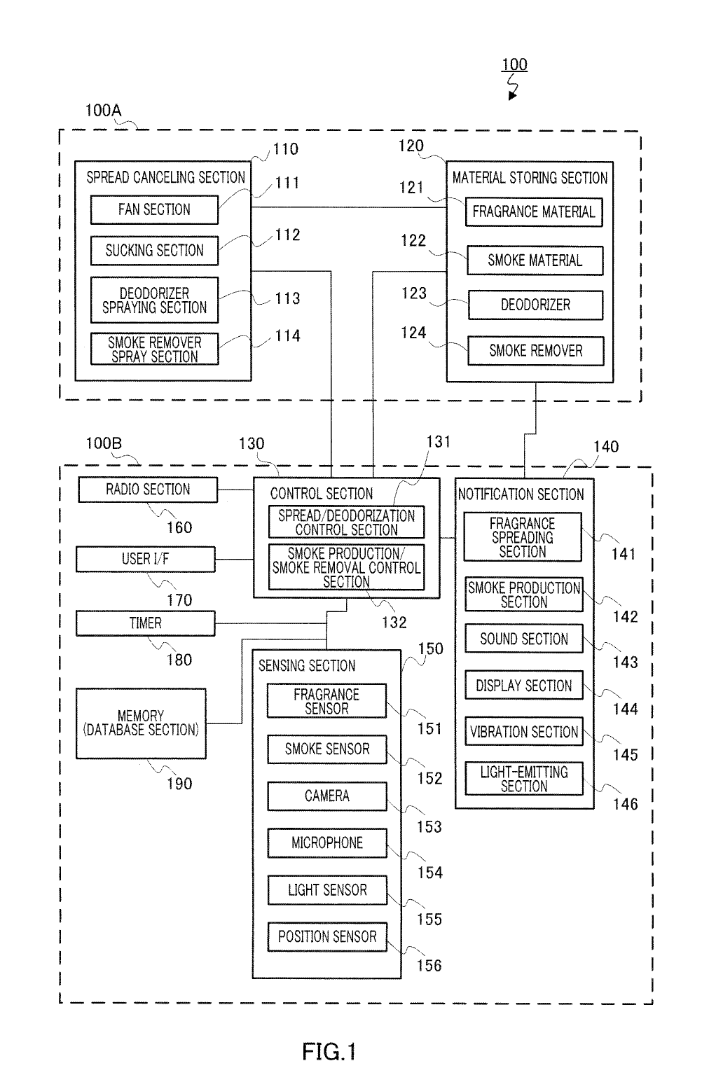

FIG. 1 shows the configuration of a communication terminal apparatus according to an embodiment of the present invention. This communication terminal apparatus 100 is mainly configured with two parts, that is, spreading apparatus body 100A that spreads fragrance, smoke and deodorizer, and mobile terminal body 100B that communicates via radio. Spreading apparatus body 100A may be integrated into mobile terminal body 100B or separate from mobile terminal body 100B. Here, in cases where spreading apparatus body 100A is apart from mobile terminal body 100B, it is desirable to enable the user to quickly recognize the spread of fragrance, for example, configuration assembling spreading apparatus body 100A into a desktop-holder-type battery charger for charging power of mobile terminal body 100B, or a configuration to enable the user to carry spreading apparatus body 100A.

Spreading apparatus body 100A in communication terminal 100 shown in the figure has spread canceling section 110 and material storing section 120. Spread canceling section 110 has fan section 111, sucking section 112, deodorizer spraying section 113 and smoke remover spray section 114, which are a means for canceling spreading fragrance. Fan section 111 has a fan that quickly blows spreading fragrance away and, removes the lasting fragrance. Sucking section 112 has a sucking device that sucks in spreading fragrance quickly, and cancels the spread of fragrance. Deodorizer spraying section 113 has a deodorizer spray opening from which a deodorizer is sprayed on fragrance, and cancels the spread of the fragrance. Smoke remover spray section 114 has a smoke remover spray opening from which a smoke remover is sprayed on spread smoke, and cancels the spread of smoke. Here, fragrance (or smoke) is removed using the removal method the user sets up and selects in advance. For example, when the user selects the method of removing lasting fragrance (smoke) with fan section 111, the blowing process is carried out by a fan. Further, when the user selects the method of removing fragrance (or smoke) by sucking section 112, a sucking device carries out the sucking process. Further, when the user selects the method of removing fragrance by deodorizer spraying section 113, the deodorizing process is carried out by issuing a spray of a deodorizer. Furthermore, when the user selects the method of removing smoke with smoke remover spray section 114, the smoke removing process is carried out by issuing a spray of a smoke remover.

Material storing section 120 has containers for storing fragrance material 121, smoke material 122, deodorizer 123 and smoke remover 124. Here, the container of fragrance material 121 is preferably a cartridge that accommodates a plurality of flavors including mint flavor, citrus flavor and aromatic flavor, and which can be replaced. Further, the containers of deodorizer 123 and smoke remover 124 are preferably gas cylinders that is sprayed on fragrance or smoke with deodorizer 123 and smoke remover 124 using nonflammable gas.

Meanwhile, mobile terminal apparatus body 100B in communication terminal apparatus 100 shown in the figures has control section 130, notification section 140, sensing section 150, radio section 160, user interface 170, timer 180 and memory 190. Control section 130 has spread/deodorization control section 131 and smoke production/smoke removal control section 132. Spread/deodorization control section 131 controls the spread of fragrance when fragrance material 121 in material storing section 120 spreads fragrance from fragrance spreading section 141 of notification section 140, and controls deodorization when deodorizer 123 in material storing section 120 is sprayed from deodorizer spraying section 113 of spread canceling section 110. Smoke production/smoke removal control section 132 controls the production of smoke when smoke material 122 in material storing section 120 produces smoke from smoke production section 142 of notification section 140, and controls smoke removal when smoke remover 124 in material storing section 120 is sprayed from smoke removal spray section 114 of spread canceling section 110.

Notification section 140 has fragrance spreading section 141, smoke production section 142, sound section 143, display section 144, vibration section 145 and light-emitting section 146, and notifies the user of an incoming call and so on using one or some of these sections. Fragrance spreading section 141 has a spreading hole from which fragrance is spread, and notifies the user of an incoming call and so on by spreading fragrance material 121 in material storing section 120 from the spreading hole into the air by a command from spread/deodorization control section 131. Smoke production section 142 has a spreading hole from which smoke is ejected, and notifies the user of an incoming call and so on by issuing a jet of smoke material 122 of material storing section 120 from the spreading hole by a command from smoke production/smoke removal control section 132. Sound section 143 notifies the user of an incoming call and so on by playing sounds and melodies (ringtones) through a speaker. Display section 144 notifies the user of an incoming call and so on by displaying information about the incoming call on a display configured with a liquid crystal panel and so on. Vibration section 145 notifies the user of an incoming call and so on by vibrating a vibrator. Light-emitting section 146 notifies the user of an incoming call and so on by blinking light-emitting elements such as LEDs.

The spreading hole from which the fragrance in fragrance spreading section 141 is spread, is configured to adjust the amount of fragrance spread or the direction of fragrance spread. For example, this spreading hole of fragrance spreading section 141, can be operated to limit the amount of fragrance spread when the amount of remaining fragrance material 121 to be spread is little and an environment related to surrounding the terminal is not suitable for spreading a large amount of fragrance. Further, this spreading hole can be operated to limit the output destination of fragrance when the direction of this fragrance spread is not preferable.

It is preferable that the color of a jet of smoke issued from the spreading hole of smoke production section 142 is white that can be colored by light-emitting diodes placed near the spreading hole. Similar to the spreading hole of fragrance spreading section 141, this spreading hole of smoke production section 142 can be operated to limit the amount of smoke when the amount of remaining smoke material 122 to produce smoke is little and the environment related to surrounding the terminal is not suitable for producing a large amount of smoke. Further, this spreading hole can be operated to limit the output destination of smoke when the direction of spreading smoke is not preferable.

Sensing section 150 has fragrance sensor 151, smoke sensor 152, camera 153, microphone 154, light sensor 155 and position sensor 156, and acquires environment information about the environment where fragrance is spread. Fragrance sensor 151 detects the types and moving direction of a smell floating near mobile terminal body 100B and sends the detection results to spread/deodorization control section 131. Smoke sensor 152 detects whether or not smoke is floating near mobile terminal body 100B, and sends the detection result to smoke production/smoke removal control section 132. Camera 153 detects whether or not the notification target user to be notified of an incoming call and so on by spreading fragrance, is near the terminal body, as to whether or not to have taken a photograph of the face of the user, and sends the detection result to control section 130. Microphone 154 detects wind power or wind direction near mobile terminal body 100B by collecting noise, and sends the detection result to control section 130. Light sensor 155 detects the open/closed state of the flip style or sliding style mobile terminal body 100B and whether or not mobile terminal body 100B is placed indoors, from the magnitude of the amount of received light, and sends the detection results to control section 130. Position sensor 156 detects the current position of mobile terminal body 100B using GPS functions, and sends the detection result to control section 130.

Radio section 160 modulates or demodulates a transmission signal transmitted from mobile terminal body 100B and a received signal received from a communicating party, to transmit and receive the signal, and sends transmitted or received information to control section 130. User interface 170 sends user setting (operation) information including mode setting and mode switching of mobile terminal body 100B, to control section 130. Timer 180 measures the timing and duration of spreading fragrance and the timing and duration of issuing a jet of smoke set up by the user in advance, and sends the measurement results to control section 130. Memory 190 stores records of spreading fragrance and jets of smoke, and sends the kept information to control section 130 according to the user's request.

Although this communication terminal apparatus 100 adopts a configuration including fragrance spreading section 141, smoke production section 142, fragrance sensor 151 and smoke sensor 152 on the mobile terminal body 100B side, these may be provided on the spreading apparatus body 100A side.

Further, the fragrance spreading method for this communication terminal apparatus 100 is as follows:
(1) The open/closed electromagnetic valve scheme whereby the spreading hole of fragrance is opened or closed with an electromagnetic valve, to spread and stop fragrance;
(2) The heat/cool scheme whereby a sheet soaked with fragrance material 121 is heated or cooled, to spread and stop fragrance;
(3) The filter paper injection scheme whereby a filter paper is soaked with fragrance material 121 through an injection needle, to spread fragrance; and
(4) The spray nozzle scheme whereby a high-pressure gas sealing cylinder with a spray nozzle accommodates fragrance material 121, and fragrance is spread by pressing the nozzle.
   Although any scheme may be implemented, in order to enable more accurate fragrance spread control, it is preferable to adopt (4) the spray nozzle scheme.

Next, the operations in communication terminal apparatus 100 of this example will be explained. With this communication terminal apparatus 100, various setting is conducted such as silent mode setting, mixed spreading setting, spreading holding setting, and mixing flavor parameter setting and timer setting.

In silent mode setting, in cases where the user is notified of an incoming call by “ringtones” or “spreading fragrance,” if these may cause trouble with others and fragrance material 121 may be consumed uselessly, the user operates setting for notifying a incoming call to use other notification methods that can prevent troubles with others and prevent fragrance material 121 from being consumed uselessly. In this communication terminal apparatus 100, this silent mode is set such that the notification setting upon receiving an incoming call for the user is switched to silent mode in which vibration section 145 drives (vibration) the vibrator and light-emitting section 146 blinks the light-emitting elements (LEDs) for example.

In mixed spreading setting, the operations in mobile terminal apparatus 100 are set up such that a fragrance mixing a plurality of fragrance materials 121 is produced and spread. For ease of explanation, cases where a fragrance is produced by using a single fragrance material 121 include here.

In spreading holding setting, when fragrance spread by the user is not possible, for example, when a decision is made that communication terminal apparatus 100 does not meet the conditions to spread fragrance, the operations in mobile terminal apparatus 100 are set up to hold the spread of fragrance. Here, whether or not communication terminal apparatus 100 meets the conditions to spread fragrance is decided depending on, for example, whether or not a silent mode is set up by user operations and whether or not environment information about mobile terminal body 100B detected in the sensors of sensing section 150 meets environmental conditions suitable for spreading fragrance. Here, the conditions where the spreading holding setting is carried out in this way, are referred to as “hold conditions.” This spreading holding setting is maintained until the silent mode setting of the above hold conditions is cleared and the environment of mobile terminal body 100B meets environment conditions suitable for spreading fragrance.

In the mixing flavor parameter setting, when a fragrance mixing a plurality of fragrance materials 121 is spread, a mixing condition (mixing parameter), that is, in what ratios a plurality of flavors selected from a plurality of fragrance materials 121 are mixed, is set up.

In the timer setting, when a fragrance spreading pattern is prepared in advance, timings and intervals of spread or hold of fragrance materials 121 corresponding to these spreading patterns are set up.

In communication terminal apparatus 100 of this example, processes such as the fragrance control requesting process, fragrance spreading process, preliminary spreading process, spreading process and fragrance spreading stop (canceling) process, are timely carried out.

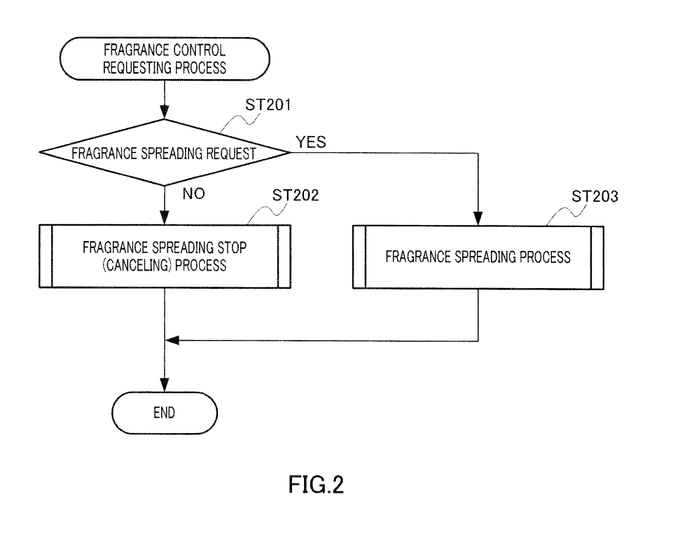

FIG. 2 shows a flow chart of the operations of the fragrance control requesting process. In this fragrance control requesting process, first, whether or not there is a fragrance spreading request is decided (step ST 201). For example, when the user sets up to notify an incoming call and so on by spreading fragrance, whether or not there is a fragrance spreading request is decided based on whether or not radio section 160 has received an incoming signal such as a phone call and a email message, that is, based on whether or not control section 130 has received an input of an incoming signal from radio section 160. When the user conducts a test for spreading fragrance, whether or not there is a fragrance spreading request is decided depending on whether or not control signal 130 receives an input of a fragrance spread test signal from user interface 170.

When a decision is made in step ST 201 that there is not a fragrance spreading request, the step moves to the fragrance spreading stop (canceling) process of canceling the fragrance spread (step ST 202). Further, when a decision is made in step ST 201 that there is a fragrance spreading request, the step moves to the fragrance spreading process of spreading fragrance (step ST 203).

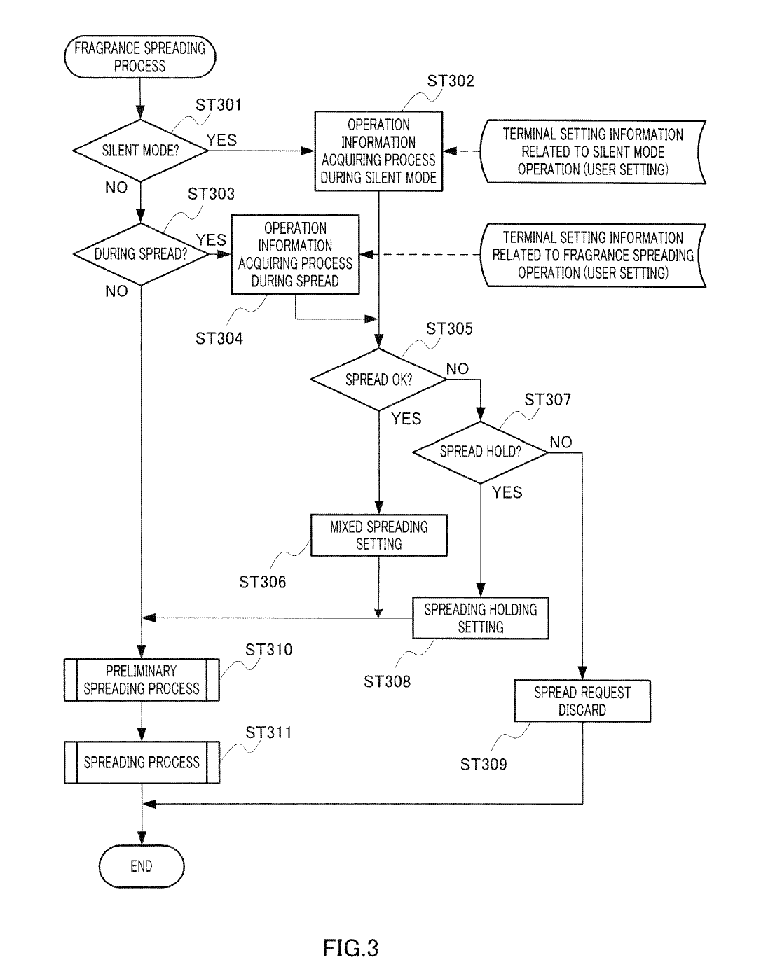

First, cases where there is a fragrance spreading request in step ST 201 and the fragrance spreading process of step ST 203 starts will be explained. FIG. 3 shows a flow chart of the operations of this fragrance spreading process.

In this fragrance spreading process, whether or not the notification setting for the user upon receiving a call in mobile terminal body 100B is the silent mode is decided (step ST 301). Here, when a decision is made that the notification setting is the silent mode, the operation information acquiring process during the silent mode is performed for acquiring terminal setting information (user settings) related to silent operations (terminal operations in the silent mode) (step ST 302).

On the other hand, when a decision is made in step ST 301 that the notification setting is not the silent mode, whether or not the fragrance spread by the previous spreading process is currently spreading is decided (step ST 303). Here, when a decision is made that the fragrance spread by the previous spreading process is currently spreading, the operation information acquiring process during spread is performed for acquiring terminal setting information (user settings) related to fragrance spreading operations (operations in the terminal upon spreading fragrance) (step ST 304).

Next, a decision is made as to whether or not fragrance spreading set by the user is possible, that is, whether or not it is permitted to spread fragrance based on the operation information acquired by the operation information acquiring process during the silent mode in step 302 or the operation information acquired by the operation information acquiring process during the spread in step ST 304 (step ST 305). Here, when a decision is made that it is permitted to spread fragrance, the operations in communication terminal apparatus 100 are set by the above mixed spreading setting so as to mix a new fragrance material 121 to be spread new and fragrance material 121 currently spreading and to spread the mixed fragrance (step ST 306).

On the other hand, when a decision is made in step ST 305 that it is not permitted to spread fragrance (i.e. it is no good to spread fragrance), whether or not to hold the spread of fragrance this time is decided (step ST 307). Here, when the user selects to hold the spread of fragrance this time, due to the above spreading holding setting, the operations in mobile terminal apparatus 100 are set up to hold the spread of fragrance this time (step ST 308). On the other hand, in step ST 307, when the user selects not to hold the spread of fragrance this time, the fragrance spreading request in step ST 201 is discarded (step ST 309) and the fragrance spreading process is finished.

Next, after the mixed spreading setting in step ST 306 and the spreading holding setting in step ST 308 are complete, or, when a decision is made in step ST 303 that the fragrance spread in the previous spreading process is not spreading, the preliminary spreading process is performed (step ST 310), and then the spreading process is performed (step ST 311) and the spreading process is finished.

In this way, with this fragrance spreading process, whether or not it is possible to spread fragrance set by the user, and whether or not communication terminal apparatus 100 spreads fragrance, are checked, and, in response to this spread request, a decision is made as to whether to spread fragrance, hold spreading fragrance, or discard the spread request.

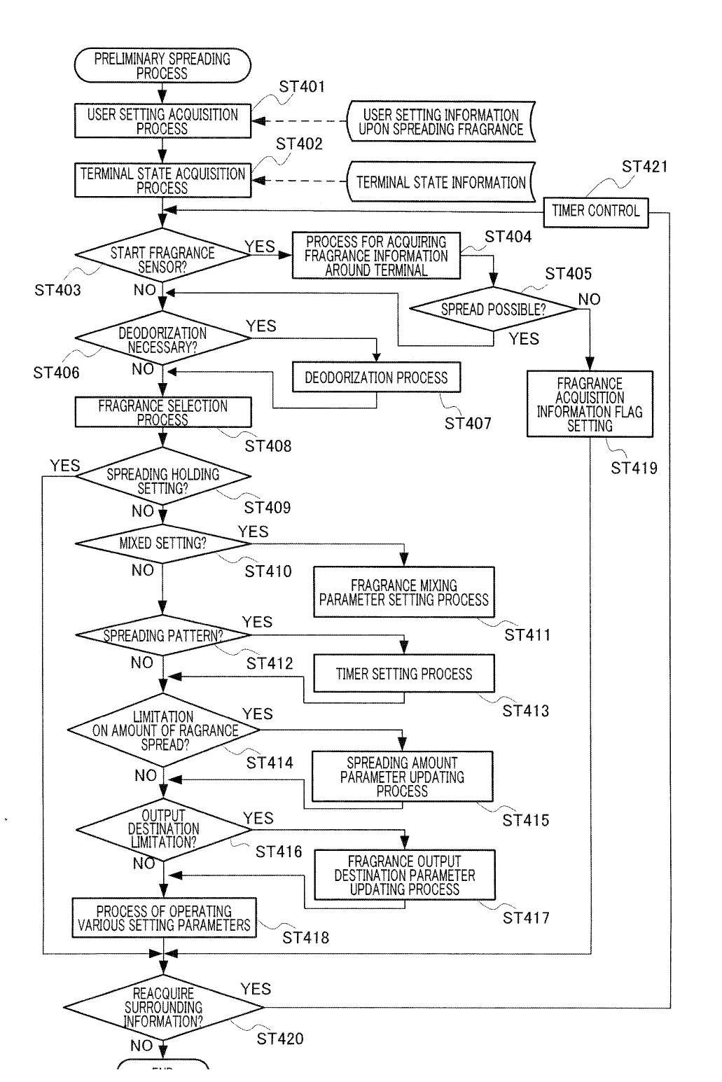

Next, the preliminary spreading process in step ST 310 will be described. FIG. 4 shows a flow chart of the operations of this preliminary spreading process. In this preliminary spreading process, first, the user setting acquisition process of acquiring user setting information upon spreading fragrance is performed (step ST 401), and next, terminal state acquisition process of acquiring terminal state information is preformed (step ST 402).

In the user setting acquisition process in step ST 401, user settings upon spreading fragrance, for example, the type of fragrance to be spread, the amount of fragrance spread, direction of fragrance spread (they are adjusted according to the angle of the spreading hole of fragrance spreading section 141) and spreading patterns of fragrance, are acquired.

Further, in the terminal state acquisition process in step ST 402, the state of communication terminal apparatus 100, for example, the style of mobile terminal body 100B (i.e. a open/closed state), the amount of remaining fragrance material 121 in material storing section 120, the environment related to surrounding mobile terminal body 100B (surrounding information) are acquired from the detection results of the sensors in sensing section 150.

Next, after the terminal state acquisition process in step ST 402 is complete, a decision is made as to whether or not fragrance sensor 151 of sensing section 150 is started (step ST 403). Here, when a decision is made that fragrance sensor 151 is started, process for acquiring the fragrance information around terminal is performed in order to acquire fragrance information including the types and moving direction of smell floating near mobile terminal body 100B (step ST 404). Then, from this fragrance information acquired in the process for acquiring the fragrance information around terminal, whether or not it is possible to spread fragrance is decided (step ST 405).

When a decision is made in step ST 405 that it is possible to spread fragrance, or when a decision is made in step ST 403 that fragrance sensor 151 does not start, whether or not it is necessary to deodorize a smell floating near mobile terminal body 100B is decided (step ST 406). Here, when a decision is made the deodorization is necessary, by operating at least one selected by the user from fan section 111, sucking section 112, deodorizer spraying section 113 of spread canceling section 110, the deodorization process of deodorizing the smell floating near mobile terminal body 100B is performed (step ST 407). Then, after this deodorization process is complete, or when a decision is made in step ST 406 that the deodorization is not necessary, the fragrance selection process of selecting the fragrance that the user has set up among a plurality of fragrance materials 121 of material storing section 120, is performed (step ST 408).

Next, after the fragrance selection process in step ST 408 is complete, whether the spreading holding setting in step ST 308 is set, is decided (step ST 409). Here, when a decision is made that the spreading holding setting is not performed, a decision is made as to whether or not to set up mixed spreading in the mixed spreading setting in step ST 306 (step ST 410).

When a decision is made in step ST 410 that mixed spreading is set, the fragrance mixing parameter setting process is performed in order to set up mixing conditions (mixing parameters), that is, in what ratios a plurality of flavors selected from a plurality of fragrance materials 121 are mixed (step ST 411). Then, after this fragrance mixing parameter setting process is complete, or when a decision is made in step ST 410 that a mixed spreading is not set, a decision is made as to whether or not there is a fragrance spreading pattern set by the user (step ST 412).

When a decision is made in step ST 412 that there is a spreading pattern, the timer setting process is performed in order to set up the timings and intervals of spread or hold of fragrance materials 121 according to this spreading pattern (step ST 413). Then, after this timer setting process is complete, or when a decision is made in step ST 412 that there is not a spreading pattern, a decision is made as to whether or not there is limitation to the amount of fragrance to be spread (step ST 414).

Here, when a decision is made in step ST 414 that there is limitation to the amount of spread, for example, the amount of remaining fragrance material 121 for spreading fragrance is little or the environment related to surrounding the terminal is not suitable for spreading a large amount of fragrance, fragrance spreading amount parameter updating process is performed in order to update the parameter of the amount of spreading fragrance (step ST 415). Then, after this fragrance spreading amount parameter updating process is complete, or when a decision is made in step ST 414 that there is no limitation to the amount of spread, whether or not destination is limited to output fragrance is decided (step ST 416).

Here, when a decision is made that the direction to spread fragrance is not preferable and there is limitation to the output destination in step ST 416, fragrance output destination parameter updating process is performed in order to update parameter of the output destination of fragrance (step ST 417). Then, after this fragrance output destination parameter updating process is complete, or when a decision is made in step ST 416 that there is no limitation on the output destination, process of operating the various setting parameters is performed in order to process operations by the above various setting parameters (step ST 418).

On the other hand, when a decision is made in step ST 405 that it is not possible to be spread, fragrance acquisition information flag setting is set in order to hold the fragrance acquisition information related to surrounding the terminal acquired in step ST 404 (step ST 419). Then, when a decision is made that there is either this fragrance acquisition information flag setting, the process of operating the various setting parameters in step ST 418, or the spreading holding setting in step ST 409, a decision is made as to whether or not to reacquire the information related to surrounding communication terminal apparatus 100 (step ST 420). Here, when a decision is made not to reacquire the surrounding information, the above preliminary spreading process is finished. On the other hand, when a decision is made to reacquire the surrounding information, timer 180 controls timings (step ST 421), to repeat, at a predetermined timing, the operations after step ST 403 to decide whether or not fragrance sensor 151 is started.

When a decision is made in step ST 405 that it is not possible to be spread, that is, when a decision is made that information related to surrounding the terminal is acquired and incoming call notification by spreading fragrance is not suitable from the acquired information, for example, in cases where the user stays at a place smelling strong, the information related to the surrounding the terminal is reacquired at arbitrary intervals and the timing to spread fragrance is in a wait, or a fragrance spreading request is discarded.

Further, when a decision is made in step ST 409 that there is spreading holding setting, the state of the terminal and information related to surrounding the terminal are acquired at arbitrary intervals until the hold condition is cleared or the spread request is discarded, and the fragrance spreading process is in a wait.

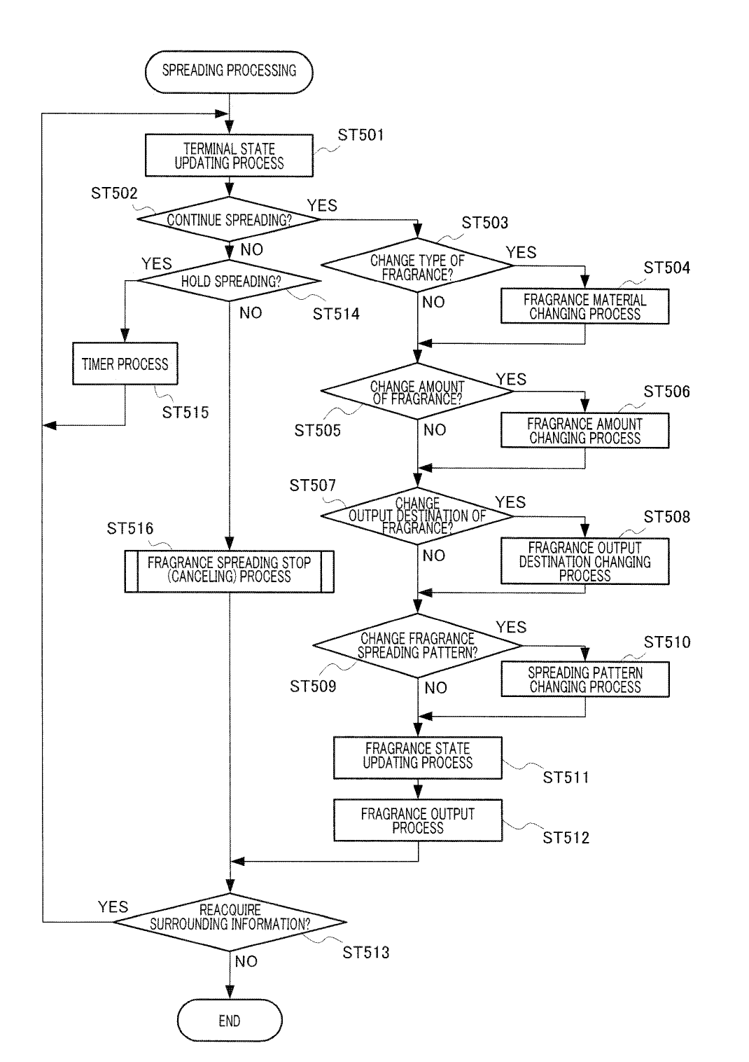

Next, the spreading process of step ST 311 will be explained. FIG. 5 shows a flow chart of the operations of the spreading process in step ST 311. In this spreading process, first, the terminal state updating process of updating the state of mobile terminal body 100B is performed (step ST 501). Then, after this terminal state updating process is complete, a decision is made as to whether or not to continue spreading fragrance (step ST 502).

When a decision is made in step ST 502 to continue spreading fragrance, a decision is made as to whether or not to change the type of the fragrance to be spread (step ST 503). Here, when a decision is made to change the type of the fragrance, the fragrance material changing process is performed in order to change the type of fragrance material 121 of material storing section 120 (step ST 504). Then, after this fragrance material changing process is complete, or when a decision is made in step ST 503 not to change the type of the fragrance, whether or not to change the amount of fragrance to be spread is decided (step ST 505).

When a decision is made in step ST 505 to change the amount of fragrance, the fragrance amount changing process of changing the amount of fragrance to be spread is performed (step ST 506). Then, after this fragrance amount changing process is complete, or when a decision is made not to change the amount of the fragrance to be spread, a decision is made as to whether or not to change the output destination of the fragrance to be spread (step ST 507).

When a decision is made in step ST 507 to change the output destination of the fragrance, the fragrance output destination changing process is performed in order to change the output destination of the fragrance to be spread (step ST 508). Then, after this fragrance output destination changing process is complete, or when a decision is made in step ST 507 not to change the output destination of fragrance, whether or not to change the fragrance spreading pattern is decided (step ST 509).

When a decision is made in step ST 509 to change the fragrance spreading pattern, the spreading pattern changing process is performed in order to change the fragrance spreading pattern (step ST 510). Then, after this spreading pattern changing process is complete, or when a decision is made in step ST 509 not to change the fragrance spreading pattern, the fragrance state updating process is performed in order to update the state of the fragrance in communication terminal apparatus 100 (step ST 511).

Next, after the fragrance state updating process in step ST 511, the fragrance output process is performed in order to output fragrance from the spreading hole in fragrance spreading section 141 of notification section 140 (step ST 512). In this fragrance output process, also during spreading of fragrance, the state of spreading fragrance and the information related to surrounding the terminal are acquired regularly, and it is possible to change the type of the fragrance, the amount of fragrance, the output destination of fragrance, the spreading pattern and so on at arbitrary timings.

Then, after the fragrance output process in step ST 512, a decision is made as to whether or not to reacquire information related to surrounding communication terminal apparatus 100 (step ST 513). Here, when a decision is made not to reacquire surrounding information, the above spreading process is finished, and, when a decision is made to reacquire the surrounding information, the step returns to the terminal state updating process in step ST 501, to repeat the above spreading process.

On the other hand, when a decision is made in step ST 502 not to continue spreading fragrance, communication terminal apparatus 100 decides whether or not to hold the spread of fragrance (step ST 514). Here, when a decision is made to hold the spread, after timer 180 performs the timer process (step ST 515), the step returns to the terminal state updating process in step ST 501, to repeat the above spreading process. The timer process in step ST 515 is not a preliminary process, and is timing control in the series of the above spreading process.

Further, when decisions is made not to continue spreading fragrance in step 502 and not to hold the fragrance spread in communication terminal apparatus 100, after the fragrance spreading stop (canceling) process is performed in order to stop (cancel) spreading fragrance (step ST 516), the step moves to step ST 513 and a decision is made as to whether or not to reacquire information related to surrounding communication terminal apparatus 100.

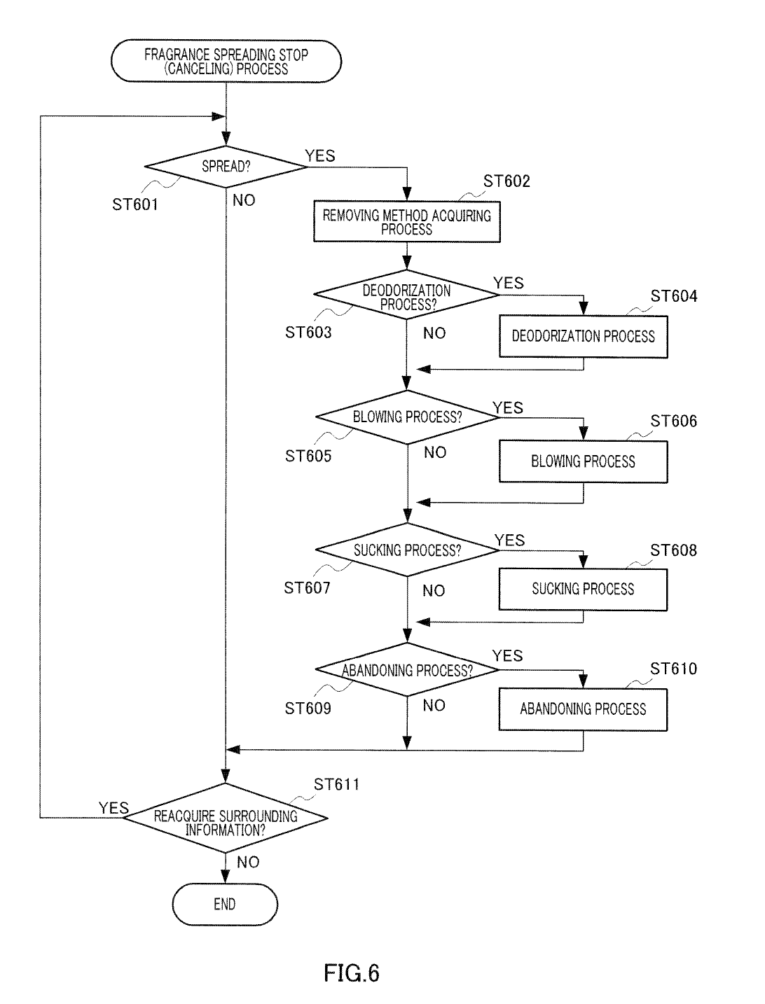

Next, the fragrance spreading stop (canceling) process in step ST 516 will be explained. FIG. 6 shows a flow chart of the operations of this fragrance spreading stop (canceling) process. In this fragrance spreading stop (canceling) process, first, whether or not to spread fragrance is decided (step ST 601). Here, when a decision is made to spread fragrance, the removing method acquiring process of selecting the method of canceling the spreading fragrance is performed (step ST 602). The remove method that can acquire in this removing method acquiring process include (1) deodorization process (2) blowing process (3) sucking process (4) abandoning process.

In this fragrance spreading stop (canceling) process, first, a decision is made as to whether or not to have selected the deodorization process in the removing method acquiring process in step ST 602 (step ST 603). Here, when a decision is made to select the deodorization process, the method of removing the fragrance by deodorizer spraying section 113 in spread canceling section 110, is selected, and the deodorization process is performed in order to spray the spreading fragrance with deodorizer 123 from the deodorizer spray opening, to deodorize the spreading fragrance (step ST 604).

Next, after the deodorization process is complete in step ST 604 or when a decision is made in step ST 603 not to select the deodorization process, a decision is made as to whether or not to have selected blowing process in the removing method acquiring process in step ST 602 (step ST 605). Here, when a decision is made to select the blowing process, the method of removing the fragrance by fan section 111 in spread canceling section 110, is selected, and the blowing process of driving the fan and blowing the spreading fragrance away, is performed (step ST 606).

Next, after the blowing process is complete in step ST 606 or when a decision is made in step ST 605 not to select the blowing process, a decision is made as to whether or not the sucking process has been selected in the removing method acquiring process in step ST 602 (step ST 607). Here, when a decision is made to select the sucking process, the method of removing the fragrance by sucking section 112 in spread canceling section 110, is selected, and the sucking process of driving a sucking device and sucking the spreading fragrance, is performed (step ST 608).

Next, after the sucking process is complete in step ST 608 or when a decision is made in step ST 607 not to select the sucking process, a decision is made as to whether or not the abandoning process has been selected in the removing method acquiring process in step ST 602 (step ST 609). Here, when a decision is made the abandoning process is selected, the removing method of waiting until the spreading fragrance fades away naturally, is selected, and the abandoning process for allowing the spreading fragrance to fade away naturally, is performed (step ST 610).

Next, after the abandoning process is complete in step ST 610, when a decision is made in step ST 609 not to select the abandoning process, or when a decision is made in step ST 601 not to spread fragrance, whether or not to reacquire information related to surrounding communication terminal apparatus 100 is decided (step ST 611). Here, when a decision is made not to reacquire surrounding information, the above fragrance spreading stop (canceling) process is finished and when a decision is made to reacquire surrounding information, the step returns to the decision in step ST 601 as to whether or not to spread fragrance, to repeat the fragrance spreading stop (canceling) process.

In this way, according to communication terminal apparatus 100 of the present example, as shown in the flow chart of the operations of the fragrance spreading process in FIG. 3, it is possible to control the spread of fragrance depending on what the request is about even when the next spread request is produced while fragrance is spreading. Accordingly, with this communication terminal apparatus 100, it is possible to cancel the mixture of the fragrance currently spreading and the fragrance to be spread new, and mix the fragrance currently spreading with the fragrance to be spread new in predetermined rules. By this means, according to this communication terminal apparatus 100, it is possible to control fragrance as follows:
(1) spreading the fragrance for the next request immediately after the fragrance currently spreading stops being spread;
(2) spreading the fragrance for the next request after the fragrance currently spreading stops being spread and an arbitrary period has passed;
(3) gradually increasing and decreasing the fragrance for the next request and the fragrance currently spreading, that is, gradually decreasing (deodorizing) the amount of fragrance currently spreading and gradually increasing the amount of spreading fragrance for the next request, to spread the fragrances; and
(4) mixing the fragrance currently spreading and the fragrance for the next request in an arbitrary ratio and spreading the mixed fragrance.

Further, with this communication terminal apparatus 100, it is possible to repeat spreading a fragrance intermittently at arbitrary intervals and cancel the spread at an arbitrary intervals while a fragrance is spread. Here, when the spread of fragrance is canceled, notification such as an incoming call is carried out by operating sound section 143, display section 144, vibration section 145 and light-emitting section 146, instead of spreading fragrance. By this means, with this communication terminal apparatus 100, it is possible to notify an incoming call and so on by “ringtones” and “vibration” even when the spread of fragrance is canceled. On the other hand, when the spread of fragrance is repeated intermittently at arbitrary intervals, after the fragrance previously spread is deodorized by releasing deodorizer 123 (smoke remover 124 in the case of smoke), the next fragrance may be spread. By this means, it is possible to prevent the fragrance previously spread and the next fragrance from mixing.

Further, with this communication terminal apparatus 100, when fragrance sensor 151 of sensing section 150 detects the fragrance around mobile terminal body 100B, this detected fragrance is compared with the spread conditions set in advance, and a decision is made that the environment of mobile terminal body 100B is not applicable to spreading fragrance, it is possible to cancel spreading fragrance. By this means, with this communication terminal apparatus 100, it is possible to cancel spreading fragrance when the fragrance concentration in the surroundings is out of a preset reference range, for example, when a strong wind blows around mobile terminal body 100B and the effect is not produced even when the fragrance is spread and the characteristics of the fragrance around mobile terminal body 100B does not match the fragrance to be spread for the next request. When the fragrance spread previously and the fragrance to be spread for the next request are not compatible, by spreading a substitute fragrance that matches the fragrance spread previously, a bad smell generated by mixing the fragrances may be prevented.

Further, with this communication terminal apparatus 100, it is possible to detect fragrance around mobile terminal body 100B in fragrance sensor 151 of sensing section 150 and control the amount of fragrance spread. By this means, with this communication terminal apparatus 100, when the concentration of fragrance around mobile terminal body 100B reaches a preset reference range, it is possible to cancel spreading fragrance automatically. Further, with this communication terminal apparatus 100, when the concentration of the fragrance around mobile terminal body 100B becomes a reference value or less, it is also possible to restart the spread of fragrance automatically.

Further, with this communication terminal apparatus 100, it is possible to detect fragrance around mobile terminal body 100B in fragrance sensor 151 of sensing section 150, issue a spray of deodorizer 123 in deodorizer spraying section 113 and deodorize the detected fragrance. By this means, with this communication terminal apparatus 100, after the fragrance detected in fragrance sensor 151 is deodorized, it is possible to spread the next fragrance automatically, and prevent the fragrance spread previously and the fragrance to be spread next from mixing. At this time, it is possible to control the period in which deodorizer spraying section 113 issues a spray of deodorizer 123 according to fragrance concentration around mobile terminal body 100B. By this means, it is possible to control the period in which the spreading fragrance is deodorized.

Further, with this communication terminal apparatus 100, it is possible to cancel a fragrance spreading request in conjunction with a specific operation such as pressing keys in user interface 170 and controls the battery level decision in mobile terminal body 100B. By this means, with this communication terminal apparatus 100, it is possible to set the “silent mode” of fragrance, for example, when fragrance sensor 151 of sensing section 150 detects a specific fragrance, it is possible to cancel spreading fragrance after that. In this case, by conducting predetermined steps of clearing by pressing specific keys and so on in user interface 170 and by passing a predetermined time using timer 180 and alarms for example, it is possible to clear the cancellation of the spread of fragrance.

Further, with this communication terminal apparatus 100, it is possible to spread a predetermined fragrance always (or during specific operations) in conjunction with remote operations and control by control section 130. By this means, for example, when mobile terminal body 100B is lost, by releasing an unpleasant smell or bad smell using remote operation and so on, it is possible to prevent others from using the lost mobile terminal body 100B.

Further, with this communication terminal apparatus 100, by setting predetermined fragrance spread conditions in advance, it is possible to change the fragrance to be spread on a temporary basis. By this means, with this communication terminal apparatus 100, for example, in cases where incoming calls are received from a single person, it is possible to spread a different fragrance from the fragrance spread previously.

Further, with this communication terminal apparatus 100, when the remaining amount of predetermined fragrance material 121 reaches a predetermined storing amount or less, it is possible to cancel spreading the fragrance or adjust the amount of fragrance to be spread. In this case, an incoming call and so on may be notified by different types of fragrances having a sufficient remaining amount, sound in sound section 143, vibration in vibration section 145, light in light-emitting section 146 and so on instead. Further, in this case, the user may select whether or not to notify an incoming call and so on using fragrance. Furthermore, in this case, information about the amount of remaining fragrance and the time fragrance material 121 is exchanged may be notified to the user by sound in sound section 143, display in display section 144, vibration in vibration section 145, light in light-emitting section 146 and so on.

Further, with this communication terminal apparatus 100, spread/deodorization control section 131 of control section 130 controls fragrance spreading section 141 of notification section 140, fan section 111, sucking section 112 and deodorizer spraying section 113 of spread canceling section 110, to control the spread and deodorization of fragrance material 121 in material storing section 120. Similar to this, smoke production/smoke removal control section 132 of control section 130 controls smoke production section 142 of notification section 140, and fan section 111 and sucking section 112 of spread canceling section 110, thereby controlling the production of smoke and the smoking removal of smoke material 122 in material storing section 120. In this way, when a user notification such as an incoming call and an alarm is controlled by producing smoke, the timings of producing smoke may be adjusted so as to synchronize the sound in sound section 143, display in display section 144, vibration in vibration section 145, light in light-emitting section 146 and so on. Further, in this case, upon spreading fragrance, the spread of fragrance may be notified by producing smoke. Furthermore, in this case, the color of smoke may vary depending on the type of fragrance to be spread.

As described above, according to communication terminal apparatus 100 of the present invention, based on the spread parameters upon spreading fragrance (or smoke) preset by the user and the state of mobile terminal body 100B and environment information related to surrounding mobile terminal body 100B held by control section 130 from detection information from sensors in sensing section 150, fragrance spreading section 141 of notification section 140 controls the spread of fragrance material 121, smoke production section 142 controls the production of smoke in smoke material 122 and spread canceling section 110 controls the removal of fragrance and smoke. With this communication terminal apparatus 100, this makes it possible to control the spread of fragrance and smoke and cancel spreading fragrance and smoke when an environment of mobile terminal body 100B is not suitable for spreading fragrance and smoke.

For example, according to this communication terminal apparatus 100, when fragrance sensor 151 detects a fragrance that is a different type of a new fragrance to be spread next, it is possible to cancel spreading the fragrance of the next request. Therefore, with this communication terminal apparatus 100, it is possible to decide whether or not it is likely to produce bad smell by mixing a plurality of different types of fragrances from the detection result in fragrance sensor 151, and prevent from producing a bad smell.

Further, according to this communication terminal apparatus 100, it is possible to cancel spreading fragrance and smoke according to the amount of light detected by light sensor 155. Therefore, with this communication terminal apparatus 100, it is possible to detect, for example, whether or not it is likely to spread fragrance and smoke uselessly in the night the user sleeps, and whether or not the spread of fragrance and smoke may cause trouble with others in a movie theater, from the amount of light detected in light sensor 155, and prevent fragrance and smoke from being spread uselessly.

Further, according to this communication terminal apparatus 100, it is possible to cancel spreading fragrance and smoke according to the position detected in position sensor 156. Therefore, with this communication terminal apparatus 100, it is possible to decide whether or not it is possible to spread fragrance and smoke from the current position of mobile terminal body 100B detected in position sensor 156 using GPS functions, and control the spread of the fragrance and smoke.

Further, when this communication terminal apparatus 100 cancels fragrance spread, it is possible to notify the user of an incoming call and so on by sound in sound section 143, vibration in vibration section 145, light in light-emitting section 146 and so on. Therefore, with this communication terminal apparatus 100, it is possible to reliably notify the user whether or not there is an incoming call, even when it is not possible to notify the user of an incoming call and so on using fragrance because the spread of the fragrance is canceled.

Further, communication terminal apparatus 100 of this example can control fragrance (or smoke) not only by using an incoming call and an alarm event, but by pressing keys (pressing arbitrary keys) in the terminal. For example, this communication terminal apparatus 100 can control the output of fragrance by pressing the key “1” in the numeric keypad.

Further, communication terminal apparatus 100 of this example can change the amount of fragrance according to the time arbitrary numeric keys are pressed. For example, this communication terminal apparatus 100 can control the spread of fragrance so as to continue spreading fragrance one second during pressing the key “2” in the numeric keypad and continue spreading fragrance five seconds by pressing the key “3” in the numeric keypad for one second. Further, the user may control the period fragrance is outputted.

Further, communication terminal apparatus 100 of this example can assign the amount of fragrance and output destination of fragrance to key numerals and control the amount and the output destination of the fragrance. For example, in the numeric keypad, this communication terminal apparatus 100 can assign the key “1” to outputting fragrance to the left, the key “3” to outputting fragrance to the right, the key “2” to output a large amount of fragrance, and the key “8” to output a small amount of fragrance.

Further, with communication terminal apparatus 100 of this example, the user can assign fragrance spreading functions (e.g. the amount of fragrance and the type of fragrance) to arbitrary keys. For example, this communication terminal apparatus 100 can stop outputting fragrance in a given time even when the user continues pressing a predetermined key.

Further, terminal apparatus body 100B in communication terminal apparatus 100 is not limited to a mobile phone that notifies an incoming call and so on using fragrance and smoke, and, may be applicable to anything that controls the spread of fragrance and smoke in spreading apparatus 100A. For example, this terminal apparatus body 100B may be applicable to personal computers, televisions, and audio devices that control the spread of an optimal fragrance and smoke suitable for video images or music, and electrical household appliances that notifies setting times in a timer using fragrance and smoke.

### INDUSTRIAL APPLICABILITY

The communication terminal apparatus according to the present invention enables suitable control to spread fragrance depending upon situations, and therefore is suitable for use in communication terminal apparatuses having fragrance spreading functions including mobile phones.

1. A communication terminal apparatus comprising:
a fragrance spreading section that spreads fragrance by a predetermined spreading request;
a spread parameter setting section that sets up a spread parameter upon spreading the fragrance by the fragrance spreading section;
an environment information acquiring section that acquires environment information about an environment in which the fragrance spreading section spreads the fragrance; and
a spread canceling section that cancels spreading the fragrance based on the spread parameter and the environment information.

2. The communication terminal apparatus according to claim 1, wherein the spread canceling section comprises a deodorizer spraying section that deodorizes spread fragrance by issuing a spray of a deodorizer.

3. The communication terminal apparatus according to claim 1, wherein:
the environment information acquiring section comprises a fragrance sensor that detects a type of the fragrance; and
the spread canceling section that stops spreading fragrance of the next spreading request, when the fragrance sensor detects a different type of the fragrance by a next spreading request.

4. The communication terminal apparatus according to claim 1, wherein:
the environment information acquiring section further comprises a light sensor that detects the amount of light in an environment in which the fragrance is spread; and
the spread canceling section that cancels spreading the fragrance according to the amount of light detected by the light sensor.

5. The communication terminal apparatus according to claim 1, wherein:
the environment information acquiring section further comprises a position sensor that detects a position of a target for spreading the fragrance; and
the spread canceling section that cancels spreading the fragrance according to the position detected by the position sensor.

6. The communication terminal apparatus according to claim 1, further comprising a spread control section that controls the spread of fragrance,
wherein the spread control section controls the fragrance spreading section such that the fragrance spreading section spreads the fragrance by the next spreading request immediately after a fragrance for a previous spreading request stops being spread.

7. The communication terminal apparatus according to claim 1, further comprising a spread control section that controls spread of the fragrance,
wherein the spread control section controls the fragrance spreading section such that the fragrance spreading section spreads the fragrance by the next spreading request after an arbitrary period passes a fragrance by the previous spreading request stops and.

8. The communication terminal apparatus according to claim 1, further comprising a spread control section that controls spread of the fragrance,
wherein the spread control section controls the fragrance spreading section such that the fragrance spreading section gradually decreases a fragrance in spreading and gradually increases the fragrance by the next spreading request.

9. The communication terminal apparatus according to claim 1, further comprising a spread control section that controls spread of the fragrance,
wherein the spread control section controls the fragrance spreading section such that the fragrance spreading section mixes a plurality of fragrances in an arbitrary ratio and spreads the mixed fragrances.

10. The communication terminal apparatus according to claim 1, further comprising a spread control section that controls spread of the fragrance,
wherein the spread control section controls the fragrance spreading section such that the fragrance spreading section repeats spreading the fragrance intermittently at arbitrary intervals.

11. The communication terminal apparatus according to claim 1, further comprising a spread control section that controls spread of the fragrance,
wherein the spread control section controls the fragrance canceling section such that the fragrance canceling section cancels spreading the fragrance in spreading at an arbitrary time.

12. The communication terminal apparatus according to claim 1, further comprising a sound section that, when a next spread is requested in a situation where the spread canceling section cancels spreading the fragrance, makes sound instead of spreading the fragrance.

13. The communication terminal apparatus according to claim 1, further comprising a vibration section that, when a next spread is requested in a situation where the spread canceling section cancels spreading the fragrance, vibrates instead of spreading the fragrance.

14. The communication terminal apparatus according to claim 1, further comprising a light-emitting section that, when a next spread is requested in a situation where the spread canceling section cancels spreading the fragrance, emits light instead of spreading the fragrance.

15. The communication terminal apparatus according to claim 1, wherein:
the environment information acquiring section comprises a fragrance concentration sensor that detects a concentration of fragrance around the terminal; and
the spread canceling section that cancels spreading the fragrance when a concentration of the fragrance detected by the fragrance concentration sensor is equal to or more than a predetermined level.

16. The communication terminal apparatus according to claim 1, further comprising a spread canceling clear section that clears a spread canceling function of fragrance by the spread canceling section.

17. The communication terminal apparatus according to claim 1, further comprising a remote control section that controls spread of the fragrance from the fragrance spreading section by a remote control.

18. The communication terminal apparatus according to claim 1, further comprising:
a fragrance material remaining amount sensor that detects the amount of remaining fragrance material spread from the fragrance spreading section; and
a fragrance spread amount adjustment section that, when the amount of remaining fragrance material detected by the fragrance material remaining amount sensor is equal to or less than a predetermined remaining amount, cancels spreading the fragrance or decreases the amount of the fragrance spread.

19. The communication terminal apparatus according to claim 1, further comprising a smoking section that produces smoke, wherein:
the spread parameter setting section sets up the spread parameter upon producing the smoke;
the environment information acquiring section comprises a smoke sensor that acquires environment information about an environment in which the smoke is produced; and
the spread canceling section cancels producing the smoke based on the spread parameter and the environment information.
